(19) United States
(12) Patent Application Publication
(10) Pub. No.: US 2026/0102102 A1
(43) Pub. Date: Apr. 16, 2026

(54) SYSTEM AND METHODS FOR LOCATING DRIVERS OF ATRIAL FIBRILLATION

(71) Applicant: CoreMap, Inc., Burlington, MA (US)

(72) Inventor: Peter S. Spector, Colchester, VT (US)

(21) Appl. No.: 19/357,922

(22) Filed: Oct. 14, 2025

Related U.S. Application Data
(60) Provisional application No. 63/707,401, filed on Oct. 15, 2024.

(51) Int. Cl. A61B 5/367; A61B 5/00; A61B 5/287; A61B 5/361; A61B 34/10

(52) U.S. Cl. CPC A61B 5/367; A61B 5/287; A61B 5/361; A61B 5/6852; A61B 5/6869; A61B 34/10; A61B 2034/105; A61B 2034/107; A61B 2560/0468; A61B 2562/227

(57) ABSTRACT

The present disclosure applies to methods of cardiac ablation that use an algorithm to determine where in the heart a driver of atrial fibrillation is located. Catheters with multi-electrode arrays are used to measure electrograms from cardiac signals wherein abnormal signals are identified as electrogram deflections with characteristic shapes. The electrogram deflections are compared to each other in pairs to determine which came first in time in order to determine the net direction of propagation of a fibrillatory wave. The signal that came first is assigned a positive point value so that once the plurality of electrograms have been compared, the signal with the highest point value can be identified as a driver of fibrillation. The data can be displayed graphically as a map of atrial fibrillation drivers on a 3D representation of a heart, allowing surgeons to quickly and easily identify where on the heart the driver is.

### BACKGROUND

Atrial fibrillation is the irregular activity of muscle fibers in the heart (fibrils) resulting in an arrhythmia, or abnormal heartbeat, associated with increased mortality and risk of stroke. Existing treatments for cardiac fibrillation include medication and surgical procedures intended to restore normal electrical activity of the atria. Medication is not always effective. When medication is not effective, clinicians may try ablation, a procedure that typically uses heat energy, cold energy, or electroporation of cells to deliberately scar or disrupt the function of targeted tissue of the heart, creating non-conducting regions of cardiac tissue that limit the propagation of irregular electrical activity.

Unfortunately, ablation is not always successful. It has been estimated that long-term success rates of ablation procedures may stand between 50 and 80%. See Calkins, 2009, Treatment of atrial fibrillation with antiarrhythmic drugs or radiofrequency ablation: two systematic literature reviews and meta-analyses, Circ Arrhythm Electrophysiol 2009 2(4):349-61, incorporated by reference. Ablation is challenging due to difficulties in identifying specific locations to ablate in cardiac tissue.

### SUMMARY OF THE INVENTION

The present invention provides methods and systems that use a cardiac mapping catheter and a “fibrillatory propagation direction algorithm to locate drivers of atrial fibrillation and thereby identify locations to ablate. Systems and methods of the invention use an array of electrodes to detect electrical activity in the heart. Each electrode records electrical activity of the heart as an electrogram. In a beating heart, electrograms will show the characteristic shapes of electrical depolarization and repolarization of myocardial cells. Each heartbeat (complete cycle of cardiac contraction and relaxation) will appear as a characteristic shape or signal across the electrograms. In the context of atrial fibrillation, multiple atrial activations are occurring for each ventricular beat, and for electrograms measured directly inside the atria, these atrial activations are themselves termed “beats.” Each atrial beat involves a wave of electrical impulses that travels across the myocardium. As the wave passes an electrode, that wave appears as a characteristic deflection on the corresponding electrogram. Each electrogram is a measure of electrical activity at one specific location over time. Because each electrogram has a time axis, one wave will appear as characteristic deflections on two electrograms in the order in which the wave reached the corresponding electrodes. Methods and systems of the invention use a large number of electrodes to measure electrical activity in numerous places throughout the heart over time. Those measurements generate data in which cardiac electrical activity appears as a large number of characteristic deflections throughout the corresponding electrograms.

Those data are provided as input to a computer-implemented fibrillatory propagation direction algorithm that will give, as output, a map or display that shows a location of the source of the aberrant electrical activity, a location referred as a driver of atrial fibrillation, or an AF driver. The mapping algorithm of the invention makes comparisons among electrograms of time and location data for characteristic deflections to identify the direction of electrical waves and to trace a wave to a location at which that wave likely originated in the data. The regular electrical activity of the healthy heartbeat, originating at the sinoatrial node, is easily recognized as such (and ignored for the purpose of locating a stable driver of atrial fibrillation). Irregular activity characteristic of atrial fibrillation appears as originating from an AF driver. The fibrillatory propagation direction mapping algorithm may identify those locations by making pair-wise comparisons between the appearances of one wave on any two electrograms. Referencing the time component of the electrogram, the algorithm may identify which electrode the wave reached first. Using spatial positioning information from the electrodes, the algorithm may identify and accumulate fibrillatory propagation directions, display them on a 3D map of the heart such that the specific locations in the cardiac tissue likely to be AF drivers can be visualized.

The electrodes may be provided by a cardiac mapping catheter. If the distal head of the catheter has some number (e.g., 44) of electrode pairs, and if the catheter is placed to make electrogram readings in some number (e.g., dozens) of places on the cardiac tissue in one procedure, and if each electrogram exhibits some number (e.g., tens to hundreds) of characteristic deflections, the input to the algorithm may include thousands, if not hundreds of thousands, of measurements of characteristic deflections in the EGM signals. The algorithm may proceed by making each pairwise comparison as the data arrive, or the implementation may be multi-threaded or parallelized for speed. The output, a map of AF driver locations, may be presented graphically, e.g., on a computer monitor, possibly showing the origin and direction of travel of the waves, or alternatively the likelihood of locations being AF drivers, superimposed on a graphical representation of the patient's heart.

The catheter head, carrying the electrodes, preferably further provides positioning and orientation data to the algorithm. This may be accomplished using magnetic positioning sensors or other fiducial markers on a portion of the catheter, optionally in conjunction with an imaging or other tracking system. The electrodes and the positioning information give the AF driver mapping algorithm time and location data for each instance of an electrode recording an event. During a mapping procedure, the surgeon will typically move the catheter around to make measurements at many locations on the heart. The connected computer, implementing the AF driver mapping algorithm, builds a 3D model of that heart using the location data. A benefit of the disclosed AF driver mapping algorithm is that it may operate in real time, allowing surgeon to view a 3D map of electrical activity of the heart during the procedure. Thus if, for example, the surgeon provisionally identifies a location that is likely to be a stable driver of atrial fibrillation, the surgeon may make additional passes, placing the electrodes at and near that location to gather additional data, which continue to be provided as input to the fibrillatory propagation direction algorithm.

The system, which includes the multiple electrodes and the connected computer system operating the fibrillatory propagation direction algorithm, provides a display that illustrates the electrical activity of the heart, which may be shown on a 3D representation of the heart. The activity may be shown using any suitable graphical convention such as sets of arrows to show conduction velocity vectors (which are straightforward to calculate with the fibrillatory propagation direction mapping data) or as a heat map (e.g., red or some other color for AF driver). That 3D representation shows the location of any driver of atrial fibrillation and may be used to guide ablation. A surgeon may navigate an ablation tool to perform ablation at or near the indicated location of the driver. Because the ablation is guided by the disclosed AF driver map, which operates in real time with a very large amount of data is input, and by the detailed display of the heart and its electrical activity, the ablation procedure has a high probability of being performed at the correct location to ablate the stable driver of atrial fibrillation. Because the ablation is well guided to the correct location, the ablation procedure has a good chance of long term success. Using systems and methods of the invention, ablation procedures will have better outcomes and better long term success rates.

In certain aspects, the invention provides a method for locating drivers of atrial fibrillation. The method includes measuring a plurality of electrograms (EGMs) from cardiac signals using electrodes on a cardiac catheter; processing the plurality of EGMs using a computer to identify a characteristic deflection in an EGM, wherein timing and location of the deflection is stored in the computer; applying an algorithm to compare deflections obtained from different electrodes and that have been determined to originate from the same wave of activation to determine which deflection occurred first in time; assigning a positive point value to the deflection that occurred first in time; repeating the comparing and assigning steps until the plurality of EGMs have been compared to each other; identifying a direction of conduction based on the deflection with the highest positive point value. The driver may be identifiable as a specific location on the heart. The method may include generating a 3D representation of the heart, wherein the positive point values for the plurality of EGMs are represented as an AF driver map on the 3D representation of the heart; displaying the 3D representation of the heart on a monitor to enable a surgeon to see a location of the driver for atrial fibrillation.

In some embodiments, the catheter comprises a multi-electrode array. The catheter may have a paddle containing a multiple electrode array. The paddle may contain between about 10 and 5,000 electrode pairs. The electrodes may be present in an array on a paddle extending from a distal portion of the catheter, and wherein the catheter includes one or more leads extending from the electrodes, through a body of catheter, to a signal processing unit. The signal processing unit may include at least one amplifier. The signal processing unit may be communicatively coupled to the computer, wherein the computer comprises a processor coupled to memory housing program instructions executable by the process to cause the computer to perform the applying, assigning, and identifying steps.

Related aspects provide a system for identifying a location of a driver of atrial fibrillation. The system includes a catheter dimensioned for insertion into a heart (e.g., via a femoral artery). The catheter has a plurality of electrodes on a distal portion of the catheter and the system includes a computer system coupled to the catheter. The computer system has a processor coupled to memory housing program instructions executable by the process to cause the computer system to: (i) process a plurality of electrograms generated by the electrodes to identify events and store in the memory, for each event: a time and a location within the heart; (ii) compare pairs of the events that are obtained from different electrodes from one wave of activation to determine which event of each pair occurred first in time; and (iii) identify a location in the heart of a driver of atrial fibrillation based on a location of at least one of the events that has an earliest time, among the events, for waves of activation that are not regular heartbeats.

The system may include a display connected to the computer system, wherein the computer system is operable to present a picture of the heart on the display with the location in the heart of the driver of atrial fibrillation shown on the picture. The plurality of electrodes may be present in an array on a paddle extending from a distal portion of the catheter. The plurality of electrodes may include pairs of stacked electrodes, wherein an axis through each pair of electrodes is substantially orthogonal to a surface of the heart when the catheter is used. The system may be installed in a catheterization laboratory, or “cath lab.”

In some embodiments, the system includes a signal processing device operably positioned between the catheter and the computer. Electrical leads from the electrodes may terminate at the signal processing device. The signal processing device may include one or more of a multiplexer/demultiplexer, an amplifier, and a filter.

The computer may operate by executing a fibrillatory propagation direction mapping algorithm stored in the memory to perform the process and compare operations. Preferably, the computer performs the process and compare operations while the catheter is generating the plurality of electrograms. In certain embodiments, the process and/or the compare operations are parallelized in multiple processors, threads, or processor cores within the computer system.

### BRIEF DESCRIPTION OF FIGURES

FIG. 1 shows a fibrillatory propagation direction map displayed on a 3D representation of a heart.

### DETAILED DESCRIPTION

Embodiments of the present invention include methods and systems for mapping and guiding the treatment of cardiac fibrillation. These methods and systems allow a clinician to minimize or prevent further episodes and increase the success rate of ablation in fibrillation patients by identifying where in the heart a driver of atrial fibrillation is located, and also determining the direction and patterns of wave propagation. Systems and methods of the disclosure use a fibrillatory propagation direction mapping algorithm for locating drivers of atrial fibrillation. A surgeon uses a cardiac catheter comprising an electrode to measure a plurality of electrograms (EGMs). The catheter is connected to a computer whereby the signals recorded by the electrodes can be recorded and stored. The catheter may include features described in US 2020/0138319 A1, incorporated by reference for all purposes. The computer can then identify which signals correspond to an event (i.e., an abnormal cardiac signal) and then apply an algorithm to compare events obtained from different electrodes. The algorithm operates by comparing events from one wave in pairs and determining which occurred first in time. The event that occurred first in time is assigned a positive point value. The algorithm compares pairs of events at least until a pattern emerges showing that one location or a small cluster of location in the heart are associated with high point values. The algorithm may even report or display the one event (and its location in the heart) that has the highest point value. The location on the heart the corresponds to the event or cluster of events with the high point value(s) is identified as the origin (e.g., driver) of fibrillation. Through the fibrillatory propagation direction mapping algorithm, it is also possible to determine the direction of conduction.

Using the signals recorded by the electrodes, and using positional information, a three-dimensional representation of the heart may be generated by the computer which displays the data graphically. In a preferred embodiment, the positive point values for the plurality of EGMs are represented as a AF driver map on the 3D representation of the heart. As an AF driver map, areas with higher point values may appear a different color or darkness or have higher “peaks”. The height of the peaks or shade of an area is relative to the data used to generate the map. A peak on the map corresponds to the location of the driver of fibrillation. The 3D representation with the AF driver map overlayed onto its surface can then be displayed on a monitor or screen to enable a surgeon to see where on the heart the driver of fibrillation is.

In an embodiment, the catheter comprises a multi-electrode array. The multi-electrode array may be on a paddle that is attached to the distal end of the catheter. The paddle may contain a number of electrodes from about 10 electrode pairs to about 5,000 electrode pairs. Each electrode generates an electrogram.

Component of an electrogram include the P wave, which represents depolarization of the atria; the QRS complex, which represents depolarization of the ventricles, and the T wave, which represents repolarization of the ventricles. During each heartbeat, a healthy heart has an orderly progression of depolarization that starts with pacemaker cells in the sinoatrial node, spreads throughout the atrium, and passes through the atrioventricular node down into the bundle of His and into the Purkinje fibers, spreading down and to the left throughout the ventricles. This orderly pattern of depolarization gives rise to the characteristic ECG tracing. To the trained clinician, an ECG conveys a large amount of information about the structure of the heart and the function of its electrical conduction system.

In the present invention, an electrode on a catheter is used in an ECG to measure a plurality of electrograms (EGMs) from cardiac signals. An electrogram is a recording of electrical activity of organs such as the brain and heart, measured by monitoring changes in electric potential (i.e., voltage). The electrode is connected to a catheter which is connected to a processor, such as a computer, to process the plurality of EGMs to identify an event. An event can be any abnormal cardiac signal (i.e., a signal that relates to an episode of cardiac fibrillation). The computer is able to identify, from the plurality of EGMs, which signals are normal cardiac rhythm, and which are abnormal. The abnormal rhythms or beats are identified, by the computer, as events. The events are then stored in the computer along with the activation times.

The computer applies an algorithm to compare the events obtained from different electrodes. In a preferred embodiment, comparisons are made between events that that are measured from one wave of activation in the cardiac tissue. The events are compared, by the computer, in pairs to determine which event occurred first in time. The event that occurred first in time may be assigned a positive point value. The steps of comparing and assigning point values may be repeated until the plurality of EGMs (i.e., events) have been compared to each other. Once all events have been compared with each other, the event with the highest positive point value is identified as a driver of fibrillation and its specific location on the heart is determined.

In a preferred embodiment, a 3D representation of the heart is generated from the recorded EGMs and their point values and displayed as a AF driver map on a 3D representation of the heart. The AF driver map depicts the point values as “peaks” with heights relative to each other. The higher the point value, the taller the “peak”. In some embodiments, the heights of the peaks are shown using a color gradient (e.g., the darker the color, the higher the peak).

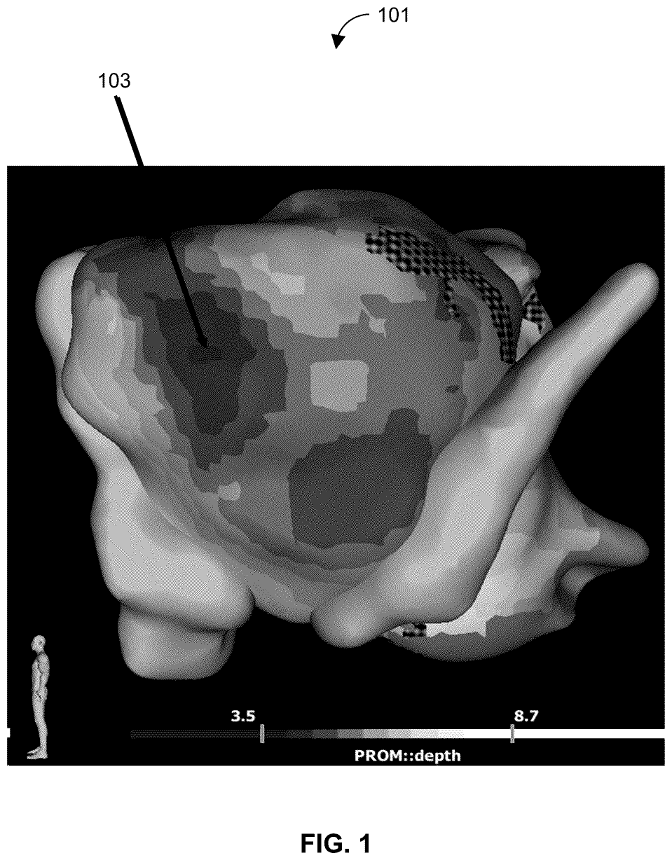

FIG. 1 shows an example of a AF driver map 101 depicted on a 3D representation of a heart. The area that is darkest 103 corresponds to a higher peak and thus a higher point value. This location also corresponds to a location of a driver of atrial fibrillation on the heart. Its graphical representation allows a surgeon or other physician to quickly and easily identify where on the heart fibrillation is originating from, allowing for quicker, easier, and more effective ablation treatment. Methods and systems of the invention use a large number of electrodes to measure electrical activity in numerous places throughout the heart over time. Those measurements generate data in which cardiac electrical activity appears as a large number of events throughout the corresponding electrograms. Those data are provided as input to a computer-implemented fibrillatory propagation direction mapping algorithm that will give, as output, a map or display that shows a location of the source of the aberrant electrical activity, a location referred as a driver of atrial fibrillation, or an AF driver. The fibrillatory propagation direction mapping algorithm of the invention makes comparisons among electrograms of time and location data for events to identify the direction of electrical waves and trace those waves to the location at which the appeared in the data. The regular electrical activity of the healthy heartbeat, originating at the sinoatrial node, is easily recognized as such. Irregular activity characteristic of atrial fibrillation appears as originating from an AF driver. The fibrillatory propagation direction algorithm may identify those locations by making pair-wise comparisons between the appearances of one wave on any two electrograms. Referencing the time component of the electrogram, the algorithm may identify which electrode the wave reached first. Using spatial positioning information from the electrodes, the algorithm may identify the specific location and direction of the wave through the cardiac tissue. The algorithm may make iterate the pairwise comparisons over pairs of electrograms until the net propagation direction of a given dwell of the multielectrode array is determined. A cumulative 3D view of the net directions of many dwells over the intracardiac surfaces can then identify locations with the atria from which waves appear to propagate and thus high likelihood of being an AF driver.

Methods and systems of the invention may use a catheter comprising a multi-electrode array on the distal end of the catheter to record cardiac signals. Such an array may have anywhere from about 10 to about 5000 electrode pairs.

An electrode is an electrical conductor. The electrodes may comprise two or more materials. For example, the electrodes may include an iridium oxide coating separated from other electrodes by one or more insulating layers. In some embodiments, individual sensing electrodes include a copper trace and a copper pad that may are plated with gold. The gold thus insulates the copper components of the electrode to prevent the copper from leaching in solution. The electrode may then be further coated with iridium oxide to provide a layer of iridium oxide to reduce the impedance and to achieve an adequate signal-to-noise ratio. The gold plating acts as a corrosion inhibitor for the copper trace. In some embodiments, the electrode comprises a slot below the insulating layer into which the copper trace is embedded such that the gold plating is on top of the copper electrode.

The electrodes may be manufactured using methods known in the art such as by using printed circuit techniques followed by coating with iridium oxide using photoresist techniques.

In some embodiments, the electrodes of the invention are microelectrodes shaped substantially as thin discs. The electrodes, as thin discs, may be positioned as a contiguous array on the surface of the paddle. The electrodes may be positioned, as shown in, for example, US 2020/0138319 A1, incorporated by reference. Notably, the height, or alternatively the thickness, of the electrode may be less than about 150 μm. In some embodiments, the height of the electrode is less than about 25 μm. Height, as used herein, means the height of the electrode as measured from the bottom of the electrode relative to its orthogonal position on the tissue to the top of the electrode. Alternatively, the height of the electrode may also refer to the thickness of the electrode as a thin disc. In non-limiting embodiments, the diameter of the electrodes may be 150 μm such that the electrode surface area is less than or equal to 0.01767 mm2.

Embodiments of the invention may use unipolar electrodes, bipolar electrodes, or orthogonal, close, unipolar (OCU) electrodes.

There are generally two electrode types: unipolar and bipolar electrodes. Unipolar electrodes have one recording electrode within the heart and another at a relatively long distance away. Unipolar electrodes are adequate but have a tendency to include far field electrical activity in the recorded signal which can result in a fractionated electrogram. Detection of far field activity may be an issue when trying to map complex arrhythmias (e.g., cardiac fibrillation) and accurately identifying local activation time. Bipolar electrode arrangements address far field activity by placing both electrodes within the heart at a relatively narrow distance apart. Since both electrodes “see” approximately the same far field electrical activity and the recorded potential is the difference of the two the resultant electrogram includes little far field signal. Certain preferred embodiments use electrode pairs that are close to each other along an axis that is substantially perpendicular to a surface of the tissue. Such orthogonal close unipolar (OCU) electrodes retain the superior near/far-field discrimination of common bipolar electrode recordings with the directional independence and smaller footprint of unipolar recordings.

Electrodes of the present disclosure may be unipolar electrodes. In a pair of unipolar electrodes, a first electrode, the “index electrode”, is proximal to tissue such that it records a signal. A second electrode, the “indifferent electrode” may be positioned away from tissue such that it does not record a signal.

Electrodes of the present disclosure may be contact bipolar electrodes. In a pair of contact bipolar electrodes, both electrodes are proximal to tissue such that each records a signal.

Electrodes of the present disclosure may be arranged as orthogonal close unipolar (OCU) electrodes. In a pair of OCU electrodes, the index electrode and indifferent electrodes are in a stacked arrangement and positioned orthogonal to a tissue. The index electrode and indifferent electrode are separated by an inter-electrode space that is likewise orthogonal to the tissue. The inter-electrode space is preferably between approximately 0.01 mm and 1 mm. In one embodiment, the index electrode can be on one flat face of the paddle while the indifferent electrode is directly opposite the index electrode on the other flat face of the paddle.

Such OCU electrodes may be particularly effective in the context of mapping fibrillation. OCU electrodes retain the ability of contact bipolar electrodes to exclude far field electrical activity endemic to unipolar arrangements. Further, OCU electrodes retain the directional independence and small footprint of unipolar electrodes, which are lacking in contact bipolar electrodes.

The electrodes used to generate electrograms may be provided on a distal portion of a cardiac mapping catheter. The electrodes may be carried by the catheter in any suitable mechanical configuration or device, such as a plurality of separate struts or arms, or as the struts or strands of a basket, or an open, mesh-like paddle. Certain embodiments of the invention include a multi-electrode array on a generally flat head or paddle. The paddle may contain from about 10 to about 5000 electrode pairs. In a preferred embodiment, the durometer of the paddle can be controlled by, for example, changing the material the paddle is made of. A non-exhaustive list of material that a paddle can be made of includes polyurethane, Teflon, polyamide, or an electroactive polymer. The electroactive polymer can change properties, such as durometer, based on an electric current that is run through it. In application, a physician can control the currents flowing through the paddle from a computer or system that is connected to the catheter while the catheter is inside the patient's heart.

Durometer can also be controlled by layering materials. A non-exhaustive list of materials that can be used for the paddle include silicone, polyimide, PEEK 450G, nylon, Pebax or Pellethane. Layers can be the same material throughout or can differ at each layer. For example, the paddle may have a first layer with PEEK or a thermoplastic polyurethane having a durometer number of 87D, and a second layer of Pebax having a durometer number of 55D.

The layers may also cover the entirety of the paddle or only some of it. For example, the second layer material may then form a web around the first material. In some embodiments, the first layer may cover one entire face of the paddle, while the second layer may form a web-like structure on top of the first layer, or vice-versa. The web structure may appear as open interstices or may resemble a web, netlike, or netted pattern.

The durometer of the paddle may also be changed by attaching to one side of the paddle (a side without electrodes) a piece of one of the above materials that is the same shape and size of the paddle. This piece of material can be attached with an adhesive and changed depending on the durometer needed for the procedure. For example, a catheter of the disclosure may be shipped in a container along with a set of peel-off adhesive-backed stiffening pieces having similar dimensions to the head or paddle of the catheter. A surgeon who prefers a stiffer (less floppy) paddle may select one of the stiffening pieces, peel off a backing to expose an adhesive, and press the stiffening piece against the back (non-electrode side) of the catheter paddle, thereby increasing a stiffness of the paddle.

Some embodiments may use temperature to ultraviolet light to modulate a stiffness or floppiness of a part of a catheter. For example, the durometer of the paddle may be changed by exposure to a certain temperature. For example, the paddle can be made with or attached to a material whose durometer is temperature dependent where warming the material can cause it to be more flexible or cooling it can make it stiffer. In another embodiment, a catheter may include a material such as a medical-grade resin that gradually stiffens as it is cured under ultraviolet (UV) light. The surgeon may remove the catheter (at full floppiness) from its package and pass the head briefly under a UV light thereby stiffening the head until the surgeon achieves her desired result.

Cardiac mapping catheters of the disclosure may include one or any number of electrodes positioned on a head or paddle of certain dimensions thereby giving the catheter a certain electrode density and consequent spatial resolution. In some embodiments, the electrode density (and spatial resolution) of the catheter may changed (e.g., adjusted or tuned), even during use. In some embodiments, the electrode density is non-uniform, allowing a surgeon to favor different portions of the head or paddle, depending on the specific task at hand.

By using an electrode array on a paddle of variable durometer, a variable recording site density can be achieved. Here, electrodes on the paddle are not evenly distributed but rather are concentrated with high density in one part of the catheter paddle and lower density at other locations on the paddle. This can be achieved by varying the durometer of the paddle in ways mentioned above. In a preferred embodiment, wires are placed in the paddle and extend out such that a physician can physically manipulate them. In doing so, the durometer can be changed based on the placement of the wires relative to each other (e.g., if the wires are closer together, then the paddle will have a lower durometer and be less taut, causing the electrodes to be less spread out and more focused i.e., dense in one area. If the wires are further apart, leading to an increase in durometer and electrodes that are more spaced out. By having a paddle with tunable conformance (through changing its durometer), there is no need to change the catheter size or shape and one can focus density in one region while simultaneously having a broad view of other areas.

What is claimed is:

1. A method for locating drivers of atrial fibrillation, the method comprising:
measuring a plurality of electrograms (EGMs) from cardiac signals using electrodes on a cardiac catheter;
processing the plurality of EGMs using a computer to identify characteristic deflections in an EGM, wherein the timing and location of the deflection is stored in the computer;
applying an algorithm to compare deflections obtained from different electrodes and that originate from the same wave of activation to determine which deflection occurred first in time;
assigning a point value to the deflection that occurred first in time;
repeating the comparing and assigning steps until the plurality of EGMs have been compared to each other;
identifying a direction of conduction based on the event with the highest positive point value.

2. The method of claim 1, wherein the event with the highest positive point value is indicative of a driver for atrial fibrillation.

3. The method of claim 2, wherein the driver is identifiable as a specific location on the heart.

4. The method of claim 1, further comprising:
generating a 3D representation of the heart, wherein the positive point values for the plurality of EGMs are represented as an AF driver map on the 3D representation of the heart;
displaying the 3D representation of the heart on a monitor to enable a surgeon to see a location of the driver for atrial fibrillation.

5. The method of claim 1, wherein the catheter comprises a multi-electrode array.

6. The method of claim 1, wherein the catheter has a paddle containing a multiple electrode array.

7. The method of claim 6, wherein the paddle contains between about 10 and 5,000 electrode pairs.

8. The method of claim 7, wherein the pairs are an index electrode and an indifferent electrode.

9. The method of claim 1, wherein the electrodes are present in an array on a paddle extending from a distal portion of the catheter, and wherein the catheter includes one or more leads extending from the electrodes, through a body of catheter, to a signal processing unit.

10. The method of claim 9, wherein the signal processing unit includes at least one amplifier and the signal processing unit is communicatively coupled to the computer, wherein the computer comprises a processor coupled to memory housing program instructions executable by the process to cause the computer to perform the applying, assigning, and identifying steps.

11. A system for identifying a location of a driver of atrial fibrillation, the system comprising:
a catheter dimensioned for insertion into a heart via a femoral artery, the catheter comprising a plurality of electrodes on a distal portion of the catheter; and
a computer system coupled to the catheter, the computer system comprising processor coupled to memory housing program instructions executable by the process to cause the computer system to
process a plurality of electrograms generated by the electrodes to identify events and store in the memory, for each event: a time and a location within the heart,
compare pairs of the events that are obtained from different electrodes from one wave of activation to determine which event of each pair occurred first in time,
identify a location in the heart of a driver of atrial fibrillation based on a location of at least one of the events that has an earliest time, among the events, for waves of activation that are not regular heartbeats.

12. The system of claim 11, further comprising a display connected to the computer system, wherein the computer system is operable to present a picture of the heart on the display with the location in the heart of the driver of atrial fibrillation shown on the picture.

13. The system of claim 11, wherein the plurality of electrodes are present in an array on a paddle extending from a distal portion of the catheter.

14. The system of claim 14, wherein the plurality of electrodes include pairs of stacked electrodes, wherein an axis through each pair of electrodes is substantially orthogonal to a surface of the heart when the catheter is used.

15. The system of claim 11, further comprising a signal processing device operably between the catheter and the computer, wherein electrical leads from the electrodes terminate in the signal processing device.

16. The system of claim 15, wherein the signal processing device comprises one or more of a multiplexer, an amplifier, and a filter.

17. The system of claim 11, wherein the computer executes a fibrillatory propagation direction mapping algorithm stored in the memory to perform the process and compare operations.

18. The system of claim 11, wherein the computer performs the process and compare operations while the catheter is generating the plurality of electrograms.

19. The system of claim 11, wherein the process and/or the compare operations are parallelized in multiple processors, threads, or processor cores within the computer system.
